(19) United States
(12) Patent Application Publication
(10) Pub. No.: US 2023/0030945 A1
(43) Pub. Date: Feb. 2, 2023

(54) SYSTEM FOR REMOVING ADDITIVE MANUFACTURING SPECIMENS FROM A BUILD PLATE

(71) Applicant: Sintavia, LLC, Hollywood, FL (US)

(72) Inventors: Keren Ott Callen, II, Plantation, FL (US); Thomas Conway Reilly, Deerfield Beach, FL (US); Tristan Keith Peyton, Tamarac, FL (US); Brody Ivan Smith, Miami, FL (US)

(73) Assignee: Sintavia, LLC, Hollywood, FL (US)

(21) Appl. No.: 17/680,785

(22) Filed: Feb. 25, 2022

Related U.S. Application Data
(60) Provisional application No. 63/227,005, filed on Jul. 29, 2021.

(51) Int. Cl. B22F 10/47; B33Y 40/20; B23H 1/00

(52) U.S. Cl. CPC B22F 10/47; B33Y 40/20; B23H 1/00

(57) ABSTRACT

The present disclosure provides a system for aiding in the removal of one or more test specimens from a build plate. The system includes an anchor portion disposed separate from the build plate. The anchor portion may include one or more attachment points for receiving and attaching one more stretchable bands. The stretchable bands may be sized to be stretched from each of the one or more specimens to the one or more of the attachment points. In this manner, a pulling force may be imparted on each test specimen, and the test specimen is moved away from the build plate during its removal. The system may be utilized particularly in conjunction with EDM and removing the test specimens using an EDM wire.

### CROSS-REFERENCE TO RELATED APPLICATION

This patent application claims the benefit of priority to U.S. Provisional Application No. 63/227,005 filed on Jul. 29, 2021, the disclosure of which is hereby incorporated by reference in its entirety and is made part of the U.S. utility application for all purposes.

### FIELD OF THE DISCLOSURE

The present development provider relates to a system for removing additive manufacturing specimens from a build plate.

### BACKGROUND OF THE DISCLOSURE

Additive manufacturing provides a process for manufacturing three-dimensional metal articles utilizing layer-by-layer formation of the article based on computer aided design (CAD) data.

Generally a metal powder is applied to a work surface, e.g., a build plate and the metal powder is subjected to one or more of a sintering, curing or melting processes. The process is repeated multiple times building an article of manufacture layer-by-layer. The resulting articles of manufacture may have complex shapes and have a wide variety of metallurgical properties. The additive manufacturing articles find utility in optimized parts in the aerospace, defense, power, oil and natural gas, and automotive industries.

The finished article, however, must be removed from the build plate. Typically, such removal has been accomplished using a cutting blade using a band saw. However such saws are not designed for that type of cutting and often leave unwanted material on the part, or on the build plate because the saw and cutting are not sufficiently exact.

Another alternative is to use electrical discharge machining (EDM). In EDM, an electric spark is created between an electrode and a conductive workpiece or part, i.e., the article of manufacture. The spark takes place in a bath of a dielectric liquid such as deionized water. The conductivity of the dielectric liquid is controlled and the water acts as a coolant and may flush away unwanted metal particles. The electric spark should be carefully controlled and localized so that very precise cuts of anything on the build plate may be removed including the article, test specimens and support structures.

EDM uses an extremely thin metallic wire to cut the articles of manufacture from the build plate. The wire is vertically oriented to the build plate. A limitation of using EDM is that care must be taken to avoid having the article of manufacture, or anything else, attached to the build plate touching the wire or bouncing off of it. If anything metal removed from the build plate comes into contact with the wire, the machine eventually short circuits. This is a particular problem when in addition to the article of manufacture being made, test specimens are also made concurrently with the article of manufacture. These test specimens are made so that various properties of the article of manufacture can be tested without having to test the article of manufacture directly. For example, testing the tensile strength of the article would destroy the actual article, but by having test specimens, these specimens may be tested for tensile strength. Thus, the article of manufacture may often be removed using EDM from the build plate and easily caught before touching the EDM wire or falling into the water bath because of the size and geometry of the article of manufacture. The test specimens, however, because of their specialized size and geometries, are more problematic.

The present disclosure provides a system for removing these test specimens from a build plate while avoiding having the removed specimen from adversely affecting the EDM wire and equipment.

### SUMMARY

To this end, the present disclosure provides a system for aiding in the removal of one or more test specimens from a build plate. The system includes an anchor portion disposed separate from the build plate. The anchor portion may include one or more attachment points for receiving and attaching one more stretchable bands. The stretchable bands may be sized to be stretched from each of the one or more specimens to the one or more of the attachment points. In this manner, a pulling force may be imparted on each test specimen, and the test specimen is moved away from the build plate during its removal. The system may be utilized particularly in conjunction with EDM and removing the test specimens by cutting and using an EDM wire.

### BRIEF DESCRIPTION OF THE DRAWINGS

Exemplary embodiments are illustrated in the referenced figures of the drawings. It is intended that the embodiment and figures disclosed herein to be illustrative rather than limiting.

FIG. 1 is a perspective view of an electric discharge machine (EDM) which may be used with a system for removal of additive manufacturing test specimens according to an embodiment.

FIG. 2 is a perspective view of the system for removal of additive manufacturing test specimens in combination with the EDM of FIG. 1 according to an embodiment.

FIG. 3 is a close-up perspective view of the system for removal of additive manufacturing test specimens shown in FIG. 2 and shows the stretchable bands according to an embodiment.

FIG. 4 is a cross-sectional view of the system.

FIG. 5 is a perspective view of the anchor portion of the system for removal of additive manufacturing test specimens according to an embodiment.

FIG. 6 is a photograph showing actual attachment of the anchor portion, the stretchable bands and several test specimens.

### DETAILED DESCRIPTION

Several embodiments will be described more fully in reference to the accompanying figure. However, this disclosure should not be construed as limited to the embodiments set forth herein. Rather, these embodiments are provided so that this disclosure will be thorough and complete, and will fully convey the scope of the invention to those skilled in the art. In the drawing, like numbers refer to like elements throughout. Thicknesses and dimensions of some components may be exaggerated for clarity.

The terminology used herein is for the purposed of describing particular embodiments only and is not intended to be limiting of the invention. As used herein, the singular forms “a,” “and,” and “the” are intended to include the plural forms as well, unless the context clearly indicates otherwise.

It will be understood that when an element is referred to as being “attached,” “coupled” or “connected” to another element, it can be directly attached, coupled or connected to the other element or intervening elements may also be present. In contrast, when an element is referred to as being “directly attached,” “directly coupled” or “directly connected” to another element, there are no intervening elements present.

All patents, patent applications and publications referred to herein are incorporated by reference in their entirety. In case of a conflict in terminology, the present specification is controlling.

It is noted that any one or more aspects or features described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

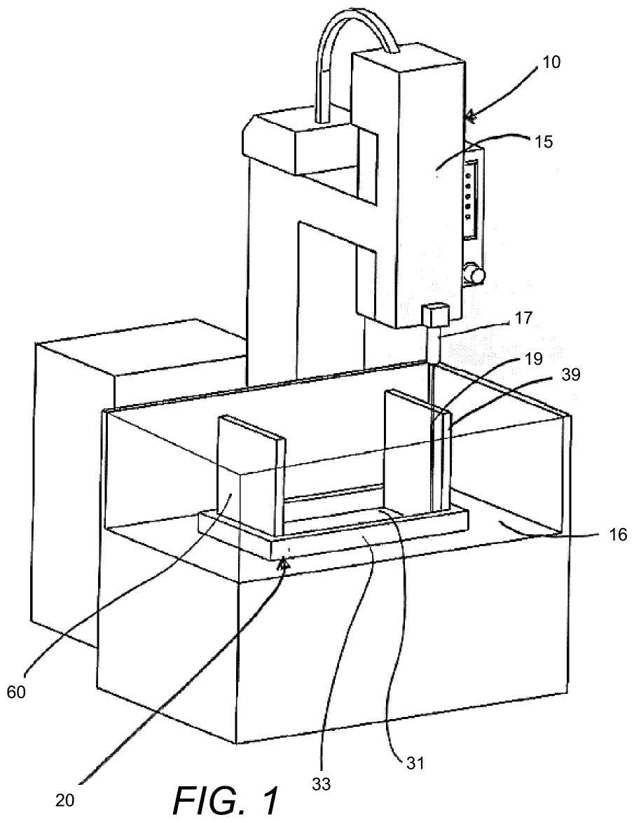

FIG. 1 is a perspective diagram of an electric discharge machine (EDM) 10. The EDM 10 may include a main machine body 15, a dielectric fluid unit 16, an upper wire guide 17, and a wire electrode 19.

Referring to FIGS. 1-3, a system 20 for aiding in the removal of additive manufacturing test specimens 35 may be mounted on a base 33 having a floor surface 31 in the dielectric fluid unit 16 of EDM 10. Also mounted on the floor surface 31 of base 33 may be the build plate 37 on a support plate 39 wherein the build plate includes the test specimens 35. The test specimens 35 are typically printed so that testing, particularly destructive testing such as tensile strength, corrosion resistance and the like may be conducted on the test specimen. The test specimen may then be discarded after the testing. Exemplary testing incudes tensile strength, harness, microhardness, tensile strain, oxidation resistance and the like based on various ASTM standards.

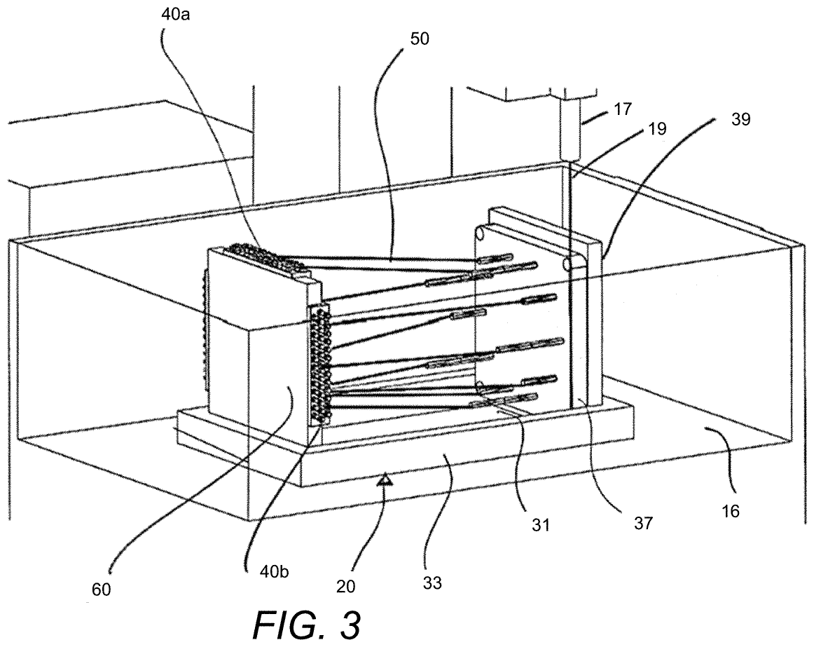

As shown in FIG. 3, the system 20 may include an anchor portion 40 separated from the build plate 37. The anchor portion 40 may include one more attachment points 45 for stretchable bands 50. The stretchable bands 50 are sized to stretch from one or more portions of the test specimens 35 to the attachment points 45. The anchor portion 40 and the surface of the build plate 37 are positioned above the height of the floor surface 31. In this manner, the stretchable bands 50 are sized to retract to a length less than that height such that the removed test specimen 15 do not adversely affect the EDM 10, for example, by striking the EDM wire electrode 19.

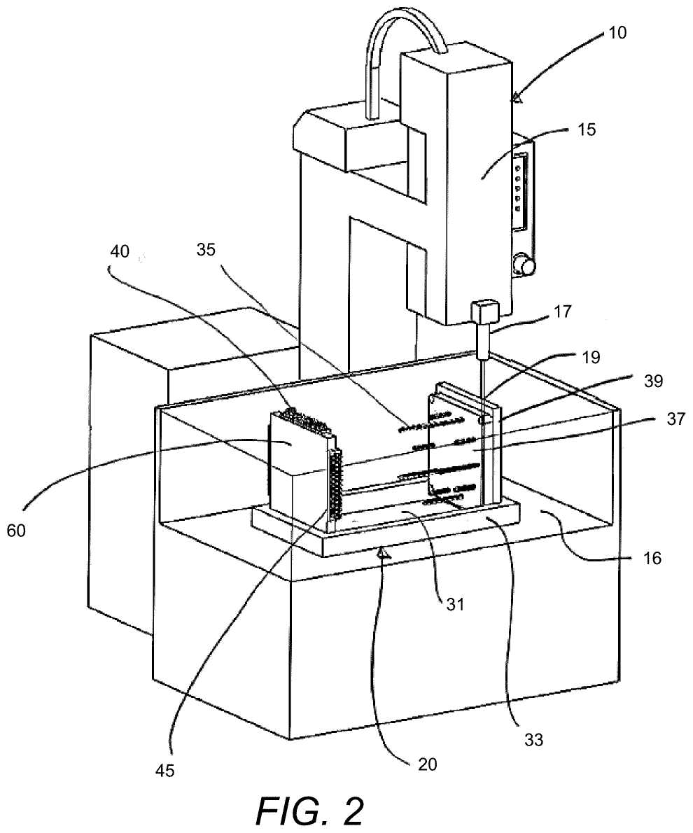

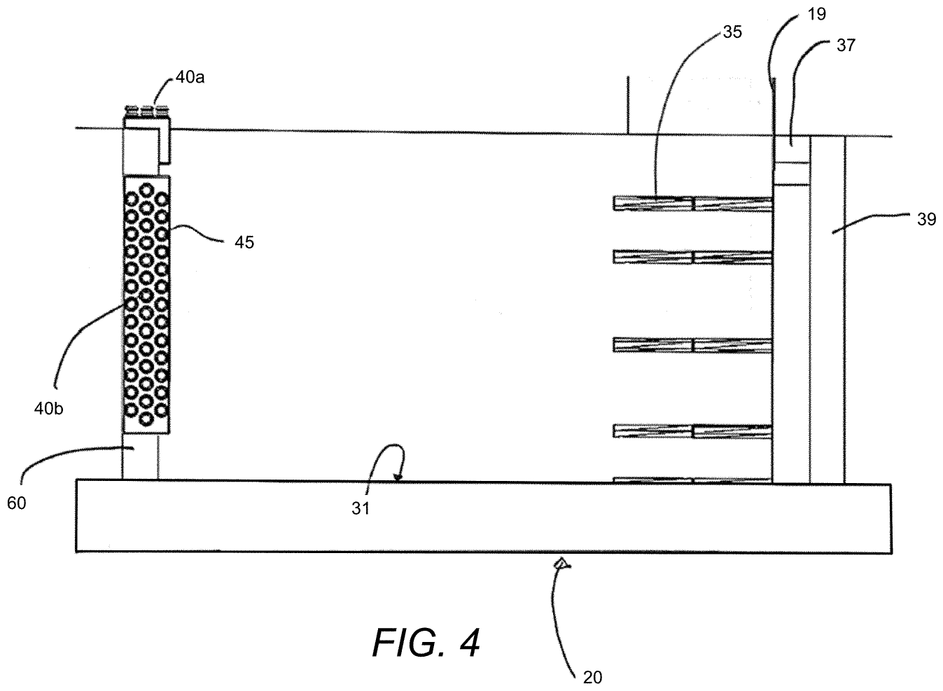

The anchor portion 40 may be mounted on an anchor plate 60 the height of which may match the build plate 37 and its support plate 39. As shown in FIGS. 2-4, the anchor portion 40 and the anchor plate 60 may be mounted vertically 40a or horizontally 40b.

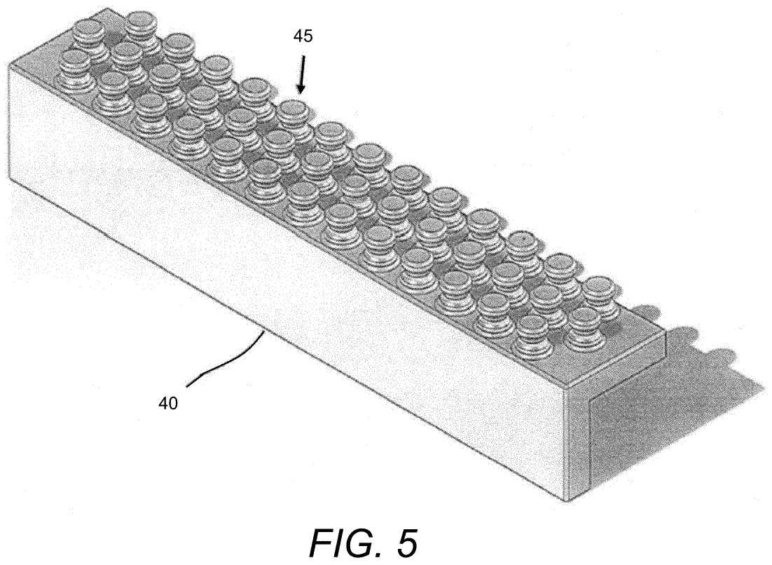

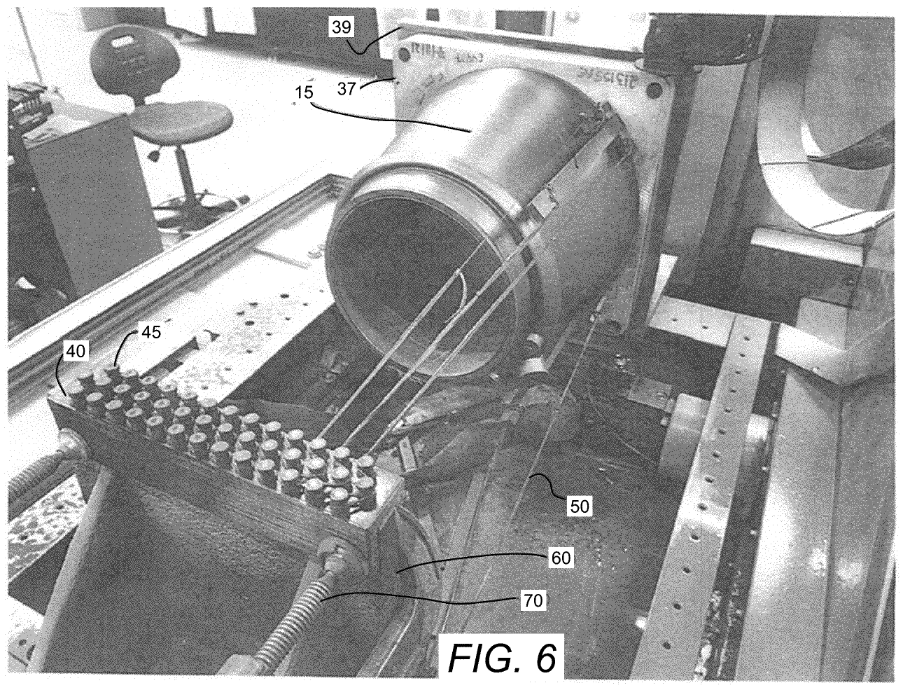

As shown in FIG. 5, the anchor portion may have multiple attachment points 45 with different channels, recesses or nub features so that the stretchable bands 50 may be wrapped around the attachment points 45 in various configurations. The anchor portion 40 may be shaped as an inverted L to facilitate attachment to the anchor plate 60. As shown in FIG. 6, the anchor portion 40 may be mounted to the top 65 or side 68 of the anchor plate 60 using a C-clamp 70 or any other attachment mechanisms.

In another embodiment, a method for removing additive manufacturing test specimens from a build plate is provided. The method includes attaching one or more stretchable bands from an anchor portion disposed separate from a build plate surface to a test specimen; and imparting a pulling force on a test specimen as the test specimen is removed from the build plate using EDM to move the test specimen away from the build plate.

It will be understood that certain features and subcombinations are of utility and may be employed without reference to other features and subcombinations and are contemplated within the scope of the claims.

That which is claimed is:

1. A system for aiding in the removal of additive manufacturing test specimens from a build plate, the system comprising:
an anchor portion disposed separate from a build plate surface, the anchor portion comprising one or more attachment points; and
one or more stretchable bands for attaching to one or more specimens to be removed from the build plate, wherein the stretchable bands are sized to be stretched from each of the one or more of the test specimens to one or more of the attachment points, thereby imparting a pulling force to move the test specimens away from the build plate as they are removed.

2. The system of claim 1, wherein the additive manufacturing test specimens are removed by cutting and utilizing EDM.

3. The system of claim 2, wherein the anchor portion and build plate surface are positioned at a height above a floor surface, and the stretchable bands are sized to retract to a length less than the height such that the removed test specimen do not adversely affect the EDM system.

4. A method for removing additive manufacturing test specimens from a build plate, the method comprising:
attaching one or more stretchable bands from an anchor portion disposed separate from a build plate surface to a test specimen; and
imparting a pulling force on a test specimen as the test specimen is removed from the build plate using EDM to move the test specimen away from the build plate.

5. The method of claim 4, wherein moving the test specimen away from the build plate avoids adversely affecting the EDM wire and equipment.
